(19) United States
(12) Patent Application Publication
(10) Pub. No.: US 2026/0007308 A1
(43) Pub. Date: Jan. 8, 2026

(54) COMPOSITE ENDOSCOPIC IMAGING DEVICE

(71) Applicant: HUKUI BIOTECHNOLOGY CO., LTD., Taipei City (TW)

(72) Inventors: SHIH-CHE LO, Taipei City (TW); YI-MING CHU, Taipei City (TW); MING-KUN CHAN, Taipei City (TW)

(73) Assignee: HUKUI BIOTECHNOLOGY CO., LTD., Taipei City (TW)

(21) Appl. No.: 18/979,555

(22) Filed: Dec. 12, 2024

Related U.S. Application Data
(60) Provisional application No. 63/667,712, filed on Jul. 4, 2024.

(30) Foreign Application Priority Data
Sep. 2, 2024 (TW) 113133048

(51) Int. Cl. A61B 1/05; A61B 1/00; A61B 1/267

(52) U.S. Cl. CPC A61B 1/05; A61B 1/00096; A61B 1/2676

(57) ABSTRACT

A composite endoscopic imaging device includes: an axial imaging device, for capturing an image in an axial direction of the composite endoscopic imaging device, the axial imaging device including: a lens, and a photosensitive element disposed on an imaging path of the lens; a flexible printed circuit board, electrically connected to the photosensitive element; and a first radial imaging device, disposed behind the axial imaging device, for capturing an image in a first radial direction of the composite endoscopic imaging device. The composite endoscopic imaging device of the present disclosure is suitably applicable in an endoscope system, and more particularly in a bronchoscope system.

### CROSS-REFERENCE TO RELATED APPLICATION

This non-provisional application claims priority under 35 U.S.C. § 119 (a) on Patent Application No(s). 113133048 filed in Taiwan, R.O.C. on Sep. 2, 2024, the entire contents of which are hereby incorporated by reference.

This non-provisional application claims priority under 35 U.S.C. § 119 (e) on US provisional Patent Application No(s). 63/667,712 filed on Jul. 4, 2024, the entire contents of which are hereby incorporated by reference.

### BACKGROUND OF THE INVENTION

### 1. Field of the Invention

The present disclosure relates to a composite endoscopic imaging device, and in particular to a composite endoscopic imaging device including an axial imaging device and a first radial imaging device.

### 2. Description of the Related Art

An endoscopic imaging device is extensively applied in inspection, diagnosis and sampling of internal organs and tissues of the human body. Different types of imaging devices have different levels of sensitivity for different lesions. Taking the lungs for example, conventional optical imaging devices, ultrasonic imaging devices, optical coherence tomography (OCT) devices and narrow-band imaging (NBI) devices have different levels of sensitivity for lesions such as inflamed tissues, malignant tumors and pulmonary nodules of the lungs.

Thus, apart from conventional optical imaging devices, ultrasonic imaging devices, OCT devices and NBI devices are also commonly used in endoscopic imaging devices to enhance accuracy of inspection, diagnosis or sampling.

However, a conventional endoscopic imaging device has only one single type of imaging device on a front end thereof. When an operator wishes to acquire different types of images, it is necessary to switch to different endoscopic devices, causing poor inspection, diagnosis or sampling efficiency.

### BRIEF SUMMARY OF THE INVENTION

There is a need for improvement for a conventional endoscopic imaging device. Therefore, it is an object of the present disclosure to provide a composite endoscopic imaging device so as to improve issues of poor inspection, diagnosis or sampling efficiency.

To achieve the above and other objects, the present disclosure further provides a composite endoscopic imaging device including: an axial imaging device, for capturing an image in an axial direction of the composite endoscopic imaging device, the axial imaging device including: a lens; and a photosensitive element, disposed on an imaging path of the lens; a flexible printed circuit board, electrically connected to the photosensitive element; and a first radial imaging device, disposed behind the axial imaging device, for capturing an image in a first radial direction of the composite endoscopic imaging device.

In one embodiment of the present disclosure, the first radial imaging device is an ultrasonic imaging device, an optical coherence tomography (OCT) device or a narrow-band imaging (NBI) device.

In one embodiment of the present disclosure, the axial imaging device further includes: a reflection device, disposed on the imaging path of the lens, wherein an upper surface of the photosensitive element is parallel to the axial direction, and the lens forms an image on the photosensitive element via the reflection device.

In one embodiment of the present disclosure, the reflection device is a mirror or a prism.

In one embodiment of the present disclosure, the reflection device is a double-sided reflection device and the first radial imaging device is an OCT device, wherein the OCT device acquires a tomography image via the double-sided reflection device.

In one embodiment of the present disclosure, the double-sided reflection device is a double-sided mirror or a double-sided reflective prism.

In one embodiment of the present disclosure, an upper surface of the photosensitive element is perpendicular to the axial direction, the lens directly forms an image on the photosensitive element, and the flexible printed circuit board bends near a side of the photosensitive element.

In one embodiment of the present disclosure, the composite endoscopic imaging device further includes: a second radial imaging device, disposed below the flexible printed circuit board, for capturing an image in a second radial direction of the composite endoscopic imaging device.

In one embodiment of the present disclosure, the second radial imaging device is an ultrasonic imaging device, an OCT device or an NBI device.

In one embodiment of the present disclosure, the second radial direction is opposite to the first radial direction.

In one embodiment of the present disclosure, the first radial imaging device is an OCT device, and the second radial imaging device is an ultrasonic imaging device.

With an axial imaging device and a first radial imaging device included, the composite endoscopic imaging device of the present disclosure enables an operator to quickly acquire different types of images, hence enhancing efficiency of inspection, diagnosis or sampling for internal organs and tissues of the human body.

### BRIEF DESCRIPTION OF THE DRAWINGS

For better clear illustration, thicknesses or sizes of the layers depicted in the drawings may be emphasized, omitted or drawn in brief. Meanwhile, the sizes of the elements do not reflected actual sizes thereof.

FIG. 1 is a schematic diagram of a composite endoscopic imaging device according to a first embodiment of the present disclosure.

FIG. 2 is a schematic diagram of a composite endoscopic imaging device according to a second embodiment of the present disclosure.

FIG. 3 is a schematic diagram of a composite endoscopic imaging device according to a third embodiment of the present disclosure.

FIG. 4 is a schematic diagram of a composite endoscopic imaging device according to a fourth embodiment of the present disclosure.

FIG. 5 is a schematic diagram of a composite endoscopic imaging device according to a fifth embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

### First Embodiment

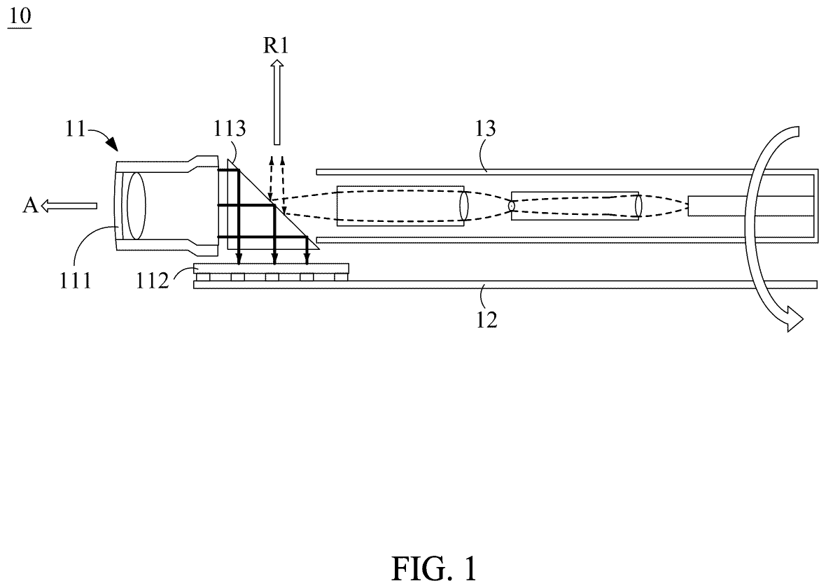

A composite endoscopic imaging device according to the first embodiment of the present disclosure is as shown in FIG. 1.

The composite endoscopic imaging device 10 according to the first embodiment of the present disclosure includes an axial imaging device 11, a flexible printed circuit board 12 and a first radial imaging device 13.

In the first embodiment, the axial imaging device 11 is for capturing an image in an axial direction A of the composite endoscopic imaging device 10, and includes: a lens 111, and a photosensitive element 112 disposed on an imaging path of the lens 111.

In the first embodiment, the axial imaging device 11 further includes a double-sided reflection device 113 disposed on the imaging path of the lens 111, wherein an upper surface of the photosensitive element 112 is parallel to the axial direction A, and the lens 111 forms an image on the photosensitive element 112 via the double-sided reflection device 113. However, the present application is not limited to the examples above. A second embodiment and a third embodiment are to be described as other examples below. In another embodiment, the axial imaging device may exclude the reflection device, or the reflection device may be a single-sided reflection device.

In the first embodiment, the flexible printed circuit board 12 is electrically connected to the photosensitive element 112, so as to transmit an image signal generated by the photosensitive element 112.

In the first embodiment, the first radial imaging device 13 is disposed behind the axial imaging device 11, and is for capturing an image in a first radial direction R1 of the composite endoscopic imaging device 10.

In one embodiment of the present disclosure, the first radial imaging device 13 is an optical coherence tomography (OCT) device; however, the present disclosure is not limited to the example above. In other embodiments, the first radial imaging device may also be an ultrasonic imaging device or a narrow-band imaging (NBI) device. In the first embodiment, apart from the lens 111 forming an image on the photosensitive element 112 via the double-sided reflection device 113, the first radial imaging device 13 (that is, the OCT device) also acquires a tomography image via the double-sided reflection device 113.

In the composite endoscopic imaging device 10 according to the first embodiment, the axial imaging device 11 and the first radial imaging device 13 share one double-sided reflection device 113, thus simplifying the structure of the composite endoscopic imaging device 10 to better promote miniaturization.

In the first embodiment, the double-sided reflection device 113 is a double-sided reflective prism, and more particularly, is a right-angle prism, of which a sloped surface has a double-sided coating layer; however, the present application is not limited to the examples above. In another embodiment, the double-sided reflection device 113 may be a double-sided mirror so as to reduce both the weight and production costs of the composite endoscopic imaging device.

The composite endoscopic imaging device 10 according to the first embodiment is suitably applicable in an endoscope system, and more particularly in a bronchoscope system. During use, an endoscope system may be guided to a lesion site by using the axial imaging device 11. Then, the composite endoscopic imaging device 10 is rotated along a direction as indicated by the arrow on the right of FIG. 1 to allow the first radial imaging device 13 to obtain a full-range image.

### Second Embodiment

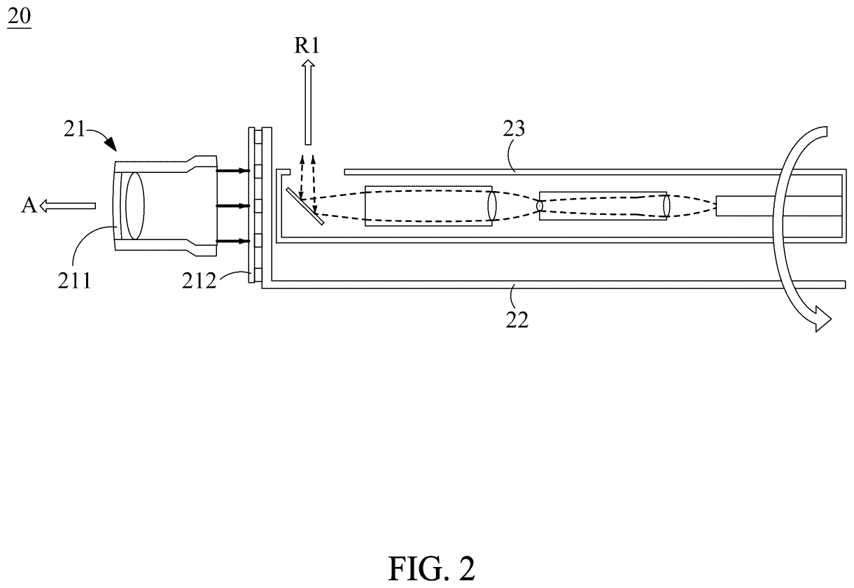

A composite endoscopic imaging device according to the second embodiment of the present disclosure is as shown in FIG. 2.

The composite endoscopic imaging device 20 according to the second embodiment of the present disclosure includes an axial imaging device 21, a flexible printed circuit board 22 and a first radial imaging device 23.

In the second embodiment, the axial imaging device 21 is for capturing an image in an axial direction A of the composite endoscopic imaging device 20, and includes: a lens 211, and a photosensitive element 212 disposed on an imaging path of the lens 211.

In the second embodiment, the flexible printed circuit board 22 is electrically connected to the photosensitive element 212, so as to transmit an image signal generated by the photosensitive element 212.

In the second embodiment, the first radial imaging device 23 is disposed behind the axial imaging device 21, and is for capturing an image in a first radial direction R1 of the composite endoscopic imaging device 20.

Compared to the first embodiment, in the composite endoscopic imaging device 20 according to the second embodiment, the axial imaging device 21 does not include any reflection device, an upper surface of the photosensitive element 212 is perpendicular to the axial direction A, the lens 221 directly forms an image on the photosensitive element 212, and the flexible printed circuit board 22 bends near a side of the photosensitive element 212.

In the composite endoscopic imaging device 20 according to the second embodiment, since the axial imaging device 21 does not include any reflection device, the structure of the composite endoscopic imaging device 20 can be simplified to better promote miniaturization.

In the composite endoscopic imaging device 20 of the second embodiment of the present disclosure, the first radial imaging device 23 is an OCT device, which has a reflection device so as to obtain a tomography image via the reflection device.

In the second embodiment, the reflection device in the OCT device is a mirror; however, the present application is not limited to the example above. In another embodiment, the reflection device in the OCT device may be a prism so as to obtain better reflection effects.

The composite endoscopic imaging device 20 according to the second embodiment is suitably applicable in an endoscope system, and more particularly in a bronchoscope system. During use, an endoscope system may be guided to a lesion site by using the axial imaging device 21. Then, the composite endoscopic imaging device 20 is rotated along a direction as indicated by the arrow on the right of FIG. 2 to allow the first radial imaging device 23 to obtain a full-range image.

### Third Embodiment

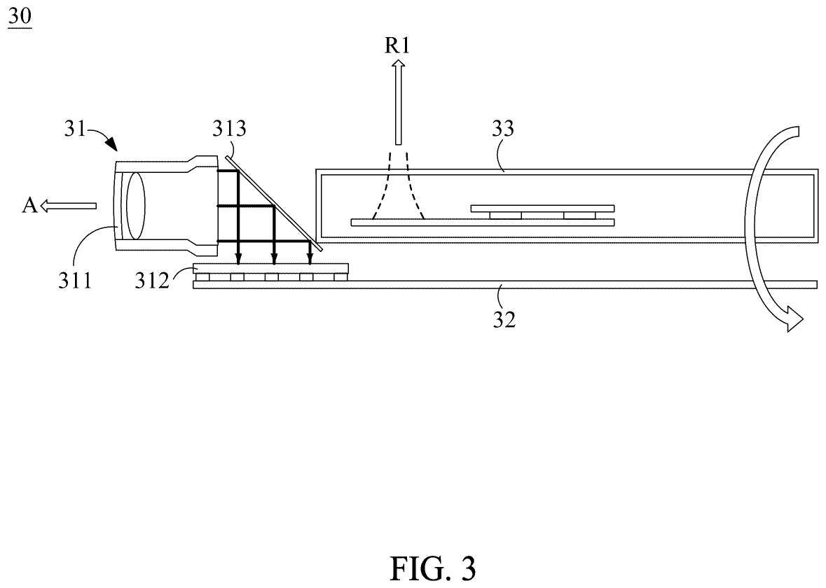

A composite endoscopic imaging device according to the third embodiment of the present disclosure is as shown in FIG. 3.

The composite endoscopic imaging device 30 according to the third embodiment of the present disclosure includes an axial imaging device 31, a flexible printed circuit board 32 and a first radial imaging device 33.

In the third embodiment, the axial imaging device 31 is for capturing an image in an axial direction A of the composite endoscopic imaging device 30, and includes: a lens 311, and a photosensitive element 312 disposed on an imaging path of the lens 311.

In the third embodiment, the flexible printed circuit board 32 is electrically connected to the photosensitive element 312, so as to transmit an image signal generated by the photosensitive element 312.

In the third embodiment, the first radial imaging device 33 is disposed behind the axial imaging device 31, and is for capturing an image in a first radial direction R1 of the composite endoscopic imaging device 30.

In the third embodiment, the axial imaging device 31 further includes a reflection device 313 disposed on the imaging path of the lens 311, wherein an upper surface of the photosensitive element 312 is parallel to the axial direction A, and the lens 311 forms an image on the photosensitive element 312 via the reflection device 313.

Compared to the first embodiment, since the third embodiment does not require any reflection device to be shared with the first radial imaging device 33, the reflection device 313 may be a single-sided reflection device.

In the third embodiment, the reflection device 313 is a mirror; however, the present application is not limited to the example above. In another embodiment, the reflection device 313 may be a prism so as to obtain better reflection effects.

Moreover, compared to the first embodiment, in the composite endoscopic imaging device 30 according to the third embodiment, the first radial imaging device 33 is an ultrasonic imaging device.

The composite endoscopic imaging device 30 according to the third embodiment is suitably applicable in an endoscope system, and more particularly in a bronchoscope system. During use, an endoscope system may be guided to a lesion site by using the axial imaging device 31. Then, the composite endoscopic imaging device 30 is rotated along a direction as indicated by the arrow on the right of FIG. 3 to allow the first radial imaging device 33 to obtain a full-range image.

### Fourth Embodiment

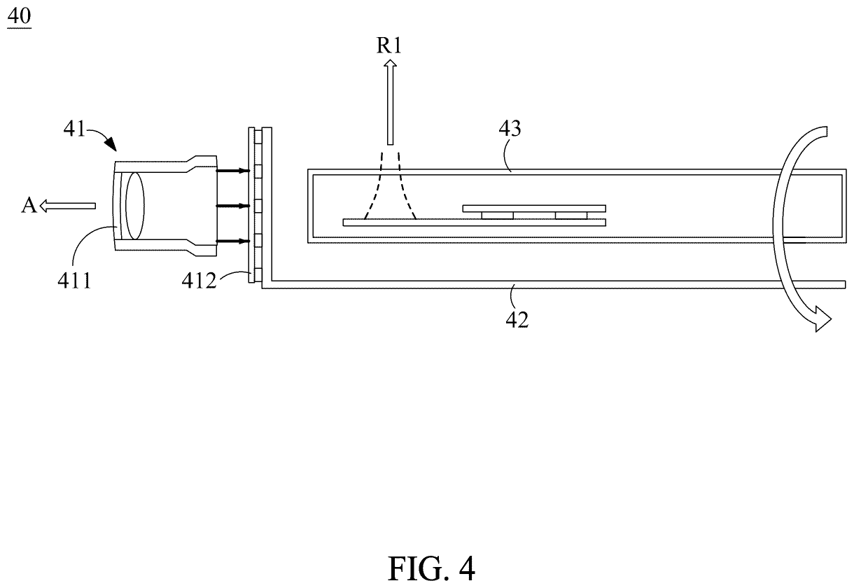

A composite endoscopic imaging device according to the fourth embodiment of the present disclosure is as shown in FIG. 4.

The composite endoscopic imaging device 40 according to the fourth embodiment of the present disclosure includes an axial imaging device 41, a flexible printed circuit board 42 and a first radial imaging device 43.

In the fourth embodiment, the axial imaging device 41 is for capturing an image in an axial direction A of the composite endoscopic imaging device 40, and includes: a lens 411, and a photosensitive element 412 disposed on an imaging path of the lens 411.

In the composite endoscopic imaging device 40 according to the fourth embodiment of the present disclosure, the axial imaging device 41 does not include any reflection device, an upper surface of the photosensitive element 412 is perpendicular to the axial direction A, the lens 421 directly forms an image on the photosensitive element 412, and the flexible printed circuit board 42 bends near a side of the photosensitive element 412.

In the composite endoscopic imaging device 40 according to the fourth embodiment, since the axial imaging device 41 does not include any reflection device, the structure of the composite endoscopic imaging device 40 can be simplified to better promote miniaturization.

In the fourth embodiment, the flexible printed circuit board 42 is electrically connected to the photosensitive element 412, so as to transmit an image signal generated by the photosensitive element 412.

In the fourth embodiment, the first radial imaging device 43 is disposed behind the axial imaging device 41, and is for capturing an image in a first radial direction R1 of the composite endoscopic imaging device 10.

The composite endoscopic imaging device 40 according to the fourth embodiment is similar to that according to the second embodiment; however, compared to the second embodiment, in the composite endoscopic imaging device 40 according to the fourth embodiment, the first radial imaging device 43 is an ultrasonic imaging device.

The composite endoscopic imaging device 40 according to the fourth embodiment is suitably applicable in an endoscope system, and more particularly in a bronchoscope system. During use, an endoscope system may be guided to a lesion site by using the axial imaging device 41. Then, the composite endoscopic imaging device 40 is rotated along a direction as indicated by the arrow on the right of FIG. 4 to allow the first radial imaging device 43 to obtain a full-range image.

### Fifth Embodiment

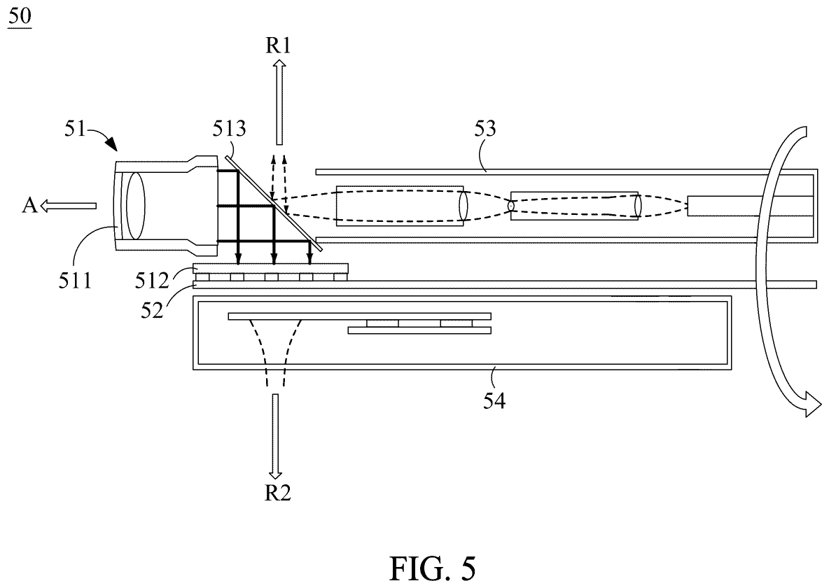

A composite endoscopic imaging device according to the fifth embodiment of the present disclosure is as shown in FIG. 5.

The composite endoscopic imaging device 50 according to the fifth embodiment of the present disclosure includes an axial imaging device 51, a flexible printed circuit board 52 and a first radial imaging device 53.

In the fifth embodiment, the axial imaging device 51 is for capturing an image in an axial direction A of the composite endoscopic imaging device 50, and includes: a lens 511, and a photosensitive element 512 disposed on an imaging path of the lens 511.

In the fifth embodiment, the axial imaging device 51 further includes a double-sided reflection device 513 disposed on the imaging path of the lens 511, wherein an upper surface of the photosensitive element 512 is parallel to the axial direction A, and the lens 511 forms an image on the photosensitive element 512 via the reflection device 513.

Similar to the first embodiment, in the composite endoscopic imaging device 50 according to the fifth embodiment, the first radial imaging device 53 is an OCT device, and the axial imaging device 1 and the first radial imaging device 53 share one double-sided reflection device 513, thus simplifying the structure of the composite endoscopic imaging device 50 to better promote miniaturization.

In the fifth embodiment, the double-sided reflection device 513 is a double-sided mirror; however, the present application is not limited to the example above. In another embodiment, the double-sided reflection device 513 may be a double-sided prism so as to obtain better reflection effects.

In the fifth embodiment, the flexible printed circuit board 52 is electrically connected to the photosensitive element 512, so as to transmit an image signal generated by the photosensitive element 512.

In the fifth embodiment, the first radial imaging device 53 is disposed behind the axial imaging device 51, and is for capturing an image in a first radial direction R1 of the composite endoscopic imaging device 50.

Compared to the first embodiment, the endoscopic imaging device 50 according to the fifth embodiment further includes: a second radial imaging device 54, disposed below the flexible printed circuit board 52, for capturing an image in a second radial direction R2 of the composite endoscopic imaging device 50.

In the composite endoscopic imaging device 50 according to the fifth embodiment, the second radial imaging device 54 is an ultrasonic imaging device; however, the present application is not limited to the example above. In another embodiment, the second radial imaging device may be an OCT device or an NBI device.

In the composite endoscopic imaging device 50 according to the fifth embodiment, the second radial direction R2 is opposite to the first radial direction R1; however, the present application is not limited to the example above. A person of ordinary skill in the art could adjust the relative orientation between the first radial direction R2 and the first radial direction R1 according to requirements.

The composite endoscopic imaging device 50 according to the fifth embodiment is suitably applicable in an endoscope system, and more particularly in a bronchoscope system. During use, an endoscope system may be guided to a lesion site by using the axial imaging device 51. Then, the composite endoscopic imaging device 50 is rotated along a direction as indicated by the arrow on the right of FIG. to allow the first radial imaging device 53 and the second radial imaging device 54 to obtain a full-range image.

With an axial imaging device and a first radial imaging device included, the composite endoscopic imaging device of the present disclosure enables an operator to quickly acquire different types of images, hence enhancing efficiency of inspection, diagnosis or sampling for internal organs and tissues of the human body.

Moreover, in one embodiment, by sharing one double-sided reflection device between the axial imaging device and the first radial imaging device, the composite endoscopic imaging device of the present disclosure can simplify the structure of the composite endoscopic imaging device to better promote miniaturization.

The present invention is described by way of the preferred embodiments above. A person skilled in the art should understand that, these embodiments are merely for describing the present invention are not to be construed as limitations to the scope of the present invention. It should be noted that all equivalent changes, replacements and substitutions made to the embodiments are to be encompassed within the scope of the present invention. Therefore, the scope of protection of the present invention should be accorded with the broadest interpretation of the appended claims.

While the present disclosure has been described by means of specific embodiments, numerous modifications and variations could be made thereto by those skilled in the art without departing from the scope and spirit of the present disclosure set forth in the claims.

What is claimed is:

1. A composite endoscopic imaging device, comprising:
an axial imaging device, for capturing an image in an axial direction of the composite endoscopic imaging device, comprising:
a lens; and
a photosensitive element, disposed on an imaging path of the lens;
a flexible printed circuit board, electrically connected to the photosensitive element; and
a first radial imaging device, disposed behind the axial imaging device, for capturing an image in a first radial direction of the composite endoscopic imaging device.

2. The composite endoscopic imaging device according to claim 1, wherein the first radial imaging device is an ultrasonic imaging device, an optical coherence tomography (OCT) device or a narrow-band imaging (NBI) device.

3. The composite endoscopic imaging device according to claim 1, wherein the axial imaging device further comprises:
a reflection device, disposed on the imaging path of the lens,
wherein an upper surface of the photosensitive element is parallel to the axial direction, and the lens forms an image on the photosensitive element via the reflection device.

4. The composite endoscopic imaging device according to claim 3, wherein the reflection device is a mirror or a prism.

5. The composite endoscopic imaging device according to claim 3, wherein the reflection device is a double-sided reflection device and the first radial imaging device is an optical coherence tomography (OCT) device, wherein the OCT device acquires a tomography image via the double-sided reflection device.

6. The composite endoscopic imaging device according to claim 1, wherein an upper surface of the photosensitive element is perpendicular to the axial direction, the lens directly forms an image on the photosensitive element, and the flexible printed circuit board bends near a side of the photosensitive element.

7. The composite endoscopic imaging device according to claim 1, further comprising:
a second radial imaging device, disposed below the flexible printed circuit board, for capturing an image in a second radial direction of the composite endoscopic imaging device.

8. The composite endoscopic imaging device according to claim 7, wherein the second radial imaging device is an ultrasonic imaging device, an optical coherence tomography (OCT) device or a narrow-band imaging (NBI) device.

9. The composite endoscopic imaging device according to claim 7, wherein the second radial imaging device is opposite to the first radial imaging device.

10. The composite endoscopic imaging device according to claim 7, wherein the first radial imaging device is an optical coherence tomography (OCT) device, and the second radial imaging device is an ultrasonic imaging device.
